(19) United States
(12) Patent Application Publication
(10) Pub. No.: US 2011/0105725 A1
(43) Pub. Date: May 5, 2011

(54) ANTIBODY PURIFICATION PROCESS

(75) Inventors: Bjarne Rønfeldt Nielsen, Virum (DK); Hanne Christensen, Frederiksberg (DK); Daniel E. Rasmussen, Kobenhavn O (DK); Thomas Budde Hansen, Kobenhavn N (DK); John Strikart Nielsen, Dragor (DK); Erik Halkjaer, Bronshoj (DK); Rikke Christina Nielsen, Frederiksberg (DK); Christina Jespersgaard, Kobenhavn V (DK); Arne Staby, Bagsvaerd (DK)

(73) Assignee: Novo Nordisk A/S

(21) Appl. No.: 12/992,661

(22) PCT Filed: May 15, 2009
(86) PCT No.: PCT/EP09/55900

§ 371 Date: Jan. 21, 2011

(30) Foreign Application Priority Data
May 15, 2008 (EP) 08156274.6

(51) Int. Cl. C07K 1/22; C07K 1/36

(52) U.S. Cl. 530/387.1

(57) ABSTRACT

The invention concerns a method for the industrial production of antibodies.

### FIELD OF THE INVENTION

The present invention relates to the purification of recombinantly expressed antibodies.

### BACKGROUND OF THE INVENTION

Processes for purifying antibodies are generally based on affinity chromatography for capture, typically Protein A, followed by ion-exchange and/or hydrophobic interaction and/or mixed mode chromatography steps. Such processes generally also include at least two virus reduction steps, typically reduction by low pH in elution from the affinity step and implementation of a virus filter in a suitable position of the process. Impurities removed by mAb purification processes include half antibodies, antibody fragments, dimers, and aggregates, DNA, vira, HCP, Protein A leakage, endotoxin and other relevant impurities.

Protein A is a 40-60 kDa surface protein originally found in the cell wall of the bacteria Staphylococcus aureus. It has found use in biochemical research because of its ability to bind immunoglobulins, most notably IgG's. It binds with the Fc region of immunoglobulins through interaction with the heavy chain.

WO9856808 and WO2005016968 describe examples of Protein A purification.

Protein A purification is also described in WO2004076485, US20070060741 and Kelley B D Biotechnol Bioeng. 101(3). 553-66 (2008).

There is a continuing need for efficient methods for the industrial production of recombinant antibodies.

### SUMMARY OF THE INVENTION

The present invention relates to a method for purifying an antibody compound from a suspension comprising said antibody compound, wherein
i) said suspension is brought into contact with a Protein A derivative/analogue under conditions, wherein the Protein A derivative/analogue binds the antibody compound,
ii) said Protein A derivative/analogue bound antibody is washed with a suitable buffer, and
iii) said antibody compound is eluted from the Protein A derivative/analogue with a suitable buffer and collected in a resulting eluate.

The present invention relates to a method for purifying an antibody compound from a suspension comprising said antibody compound, wherein
i) said suspension is brought into contact with ligand having affinity for such antibody under conditions, wherein said ligand binds the antibody compound,
ii) said ligand bound antibody is washed with a suitable buffer,
iii) said antibody compound is eluted from the ligand with a suitable buffer and collected in an eluate, and
   the eluate from step iii) is subjected to cation chromatography.

The present invention relates to an antibody purifying platform, which platform comprises a method according to the invention.

The present invention relates to a process for the industrial production of antibodies, wherein said process comprises a method according to the invention.

### DETAILED DESCRIPTION OF THE INVENTION

General methods for purification of antibodies are wellknown in the art and are for instance described in Pete Gagnon: Purification Tools for monoclonal Antibodies (1996) ISBN-9653515-9-9. This invention is concerned with the delopment of new methods for purification of antibody compounds. In the context of this application, an antibody compound is an immuglobulin. The term “immunoglobulin” refers to a molecule belonging to a class of structurally related glycoproteins consisting of two pairs of polypeptide chains, one pair of light (L) chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain is typically comprised of a heavy chain variable region (V_{H}) and a heavy chain constant region (typically comprising three domains, C_{H}1, C_{H}2, and C_{H}3). Each light chain typically is comprised of a light chain variable region (V_{L}) and a light chain constant region (typically comprising one domain, C_{L}).

The term “antibody compound” in the context of the present invention refers to an immunoglobulin molecule, a fragment of an immunoglobulin molecule, or a derivative of either thereof, which has the ability to specifically bind to an antigen under typical physiological conditions for significant periods of time such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen).

The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and the first component (Clq) of the classical complement system.

As indicated above, the term antibody herein, unless otherwise stated or clearly contradicted by context, includes fragments of any suitable full length antibody, which fragment retains the ability to specifically bind to an antigen, and which can be bound by Protein A, which also makes it capable of binding to a Protein A derivative/analogue.

Antibodies may have other structures than the “typical” immunoglobulin structure as described above. It could be a single-chain antibody, diabodies, and all kinds of fragments and combinations of light and heavy chains. The litterature is filled with description of such antibodies. For the purpose of the present invention, it is sufficient that the antibody compound binds to Protein A and an antigen.

In one embodiment, the antibody compound is a therapeutic antibody.

In one embodiment, the antibody compound is an IgG antibody.

In one embodiment, the present invention provides methods for purification of antibody compounds, which method comprises the use of affinity chromatography based on a Protein A derivative/analogue in contrast to conventional Protein A columns. Such Protein A derivative/analogue columns provide less ligand leakage, which improves running production costs and improves product quality. Further, elution conditions employed with less salt compared to traditional Protein A steps results in avoidance of any concentration steps, such as UF/DF, between affinity and ion-exchange steps.

In one embodiment, the present invention provides a method for purifying an antibody compound from a suspension comprising said antibody compound, wherein
i) said suspension is brought into contact with a Protein A derivative/analogue under conditions, wherein the Protein A derivative/analogue binds the antibody compound,
ii) said Protein A derivative/analogue bound antibody is washed with a suitable buffer, and
iii) said antibody compound is eluted from the Protein A derivative/analogue with a suitable buffer and collected in a resulting eluate.

In the context of the present invention, a Protein A derivative/analogue is a Protein A derivative/analogue ligand, wherein alkali-labile amino acids in the IgG binding domain of Protein A has been replaced with more alkali-stable amino acids. In one embodiment, a Protein A derivative/analogue is a Protein A molecule, wherein one or more asparagine (Asn) residies have been modified to increase protein stability under alkaline conditions. In one embodiment, two or more Asn residues are modified. In one embodiment, all Asn residues have been modified. In one embodiment, said Asn residues have been replaced with an amino acid selected from lysine, aspartic acid and leucine. In one embodiment, the protein A derivative/analogue is domain Z modified as described herein before. In one embodiment, the Protein A derivative/analogue is a Protein A derivative/analogue as described in EP1123389A1 and/or in U.S. Pat. No. 6,831,161. The parent Protein A molecule can also have been modified in other ways, which may for instance increase performance.

In one embodiment, said Protein A derivative/analogue is covelently attached to an inert resin. In one embodiment, said Protein A derivative/analogue resin is MabSelect SuRe™ MabSelect SuRe™ is available from GE Healthcare life Sciences (http://www.gelifesciences.com).

In one embodiment, the affinity chromatography using a Protein A derivative/analogue is performed at a temperature below room temperature. In one embodiment, the affinity chromatography using a Protein A derivative/analogue is performed at a temperature of from 2 to 25° C., such as from 5 to 25° C., for instance from 10 to 25° C., such as from 15 to 25° C., or at a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C., or at a temperature of from 2 to 10° C., such as from 5 to 10° C. or at a temperature of from 2 to 5° C. In one embodiment, the affinity chromatography using a Protein A derivative/analogue is performed at a temperature of 2, 5, 10, 15, 20 or 25° C.

In one embodiment, steps i), ii) and iii) may be performed as flow-through using a membrane or a solid resin.

In one embodiment, the eluate from the Protein A derivative/analogue chromotography is subjected to virus inactivation. In one embodiment, said virus inactivation is performed by lowering the pH of the eluate from the Protein A derivative/analogue chromotography. In one embodiment, the pH of the eluate is lowered to a pH of from 3 to 4 for a period of from 5 minutes to a day. In one embodiment, the pH is lowered to a pH of from 3.1 to 4, for instance from 3.2 to 4, such as from 3.3 to 4, for instance from 3.4 to 4, such as from 3.5 to 4, for instance from 3.6 to 4, such as from 3.7 to 4, for instance from 3.8 to 4, such as to a pH of 4. In one embodiment, the pH is lowered to a pH of from 3 to 3.9, such as from 3.1 to 3.9, for instance from 3.2 to 3.9, such as from 3.3 to 3.9, for instance from 3.4 to 3.9, such as from 3.5 to 3.9, for instance from 3.6 to 3.9, such as from 3.7 to 3.9, for instance to a pH of 3.9. In one embodiment, the pH is lowered to a pH of from 3 to 3.8, such as from 3.1 to 3.8, for instance from 3.2 to 3.8, such as from 3.3 to 3.8, for instance from 3.4 to 3.8, such as from 3.5 to 3.8, for instance from 3.6 to 3.8, such as to a pH of 3.8. In one embodiment, the pH is lowered to a pH of from 3 to 3.7, such as from 3.1 to 3.7, for instance from 3.2 to 3.7, such as from 3.3 to 3.7, for instance from 3.4 to 3.7, such as from 3.5 to 3.7, for instance to a pH of 3.7. In one embodiment, the pH is lowered to a pH of from 3 to 3.6, such as from 3.1 to 3.6, for instance from 3.2 to 3.6, such as from 3.3 to 3.6, for instance from 3.4 to 3.6, such as to a pH of 3.6. In one embodiment, the pH is lowered to a pH of from 3 to 3.5, such as from 3.1 to 3.5, for instance from 3.2 to 3.5, such as from 3.3 to 3.5, for instance to a pH of 3.5. In one embodiment, the pH is lowered to a pH of from 3 to 3.4, such as from 3.1 to 3.4, for instance from 3.2 to 3.4, such as to a pH of 3.4. In one embodiment, the pH is lowered to a pH of from 3 to 3.3, such as from 3.1 to 3.3, for instance to a pH of 3.3. In one embodiment, the pH is lowered to a pH of from 3 to 3.2, such as to a pH of 3.2, or to a pH of 3.1 or to a pH of 3.

As stated above, the eluate from the Protein A derivative/analogue chromotography may be kept at either for these pH values from a period of from 5 minutes to a day. In one embodiment, this period is from 10 minutes to 240 minutes, such as for instance from 20 to 90 minutes.

Afterwards, the pH of the eluate is adjusted to a pH of from 4.5 to 5.5 or as otherwise appropriate for any following steps.

In one embodiment, the eluate from the Protein A derivative/analogue chromotography is filtered prior to said lowering of the pH value and/or after the readjustment of pH.

In one embodiment of the present invention, the resulting eluate from the Protein A derivative/analogue chromatography, whether subjected to a pH lowering as described above or not, is subjected to a cation-exchange step. Placing a cation exchange step downstream of the affinity step may be advantageous in that the pH of the resulting eluate is lower than 7 and the eluate may be processed without further adjustment and potential additional pH adjustments, which may cross the isoelectric point of the mAb. The avoidance of further pH adjustments may assist in avoiding precipitation and aggregate formation.

The cation chromatography may be performed by loading the eluate from the protein A (possibly after the virus inactivation) pool on the column pre-equilibrated in Sodium acetate buffer at pH 4.5-6.0. Unbound material is washed out of the column and the mAb is eluted using a linear gradient from 0-0.3 M sodium chloride in a sodium acetate buffer. Aggregates is eluted as peak after the product. Impurities such as host cell proteins, DNA and leakage of Protein A is also reduced significantly.

In one embodiment, the cation chromatography is performed at a temperature below room temperature. In one embodiment, the cation chromatography is performed at a temperature of from 2 to 25° C., such as from 5 to 25° C., for instance from 10 to 25° C., such as from 15 to 25° C., or at a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C., or at a temperature of from 2 to 10° C., such as from 5 to 10° C. or at a temperature of from 2-5° C. In one embodiment, the cation chromatography is performed at a temperature of 2, 5, 10, 15, 20 or 25° C.

In one embodiment, the cation chromatography is performed as flow-through using a membrane or a solid resin.

In run through mode, the column or membrane is equilibrated in sodium acetate buffer at pH 4.5-6.0. The column is loaded untill an unacceptable increase in either HCP (host cell proteins), aggregates or other impurities is present in the collected pool (sample/product fraction).

In one embodiment, a virus filtration is performed after the cation chromatography. In one embodiment, the virus filtration is performed at a temperature below room temperature. In one embodiment, the virus filtration is performed at a temperature of from 2 to 25° C., such as from 5 to 25° C., for instance from 10 to 25° C., such as from 15 to 25° C., or at a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C., or at a temperature of from 2 to 10° C., such as from 5 to 10° C. or at a temperature of from 2-5° C. In one embodiment, the virus filtration is performed at a temperature of 2, 5, 10, 15, 20 or 25° C.

Virus filtration can be done as known in the art for instance using virus filters, for instance as described in Pete Gagnon: Purification Tools for monoclonal Antibodies (1996) ISBN-9653515-9-9.

In one embodiment, the virus filtration step is repeated, for instance using a similar virus filter or a different virus filter for the second virus filtration. In one embodiment, the first filter is more porous than the second. This enables a more efficient removal of aggrates in the first step and a subsequent more efficient removal of virus in the second step.

In one embodiment, the eluate from the cation chromatography may be subjected to anion chromatography, possibly after a virus filtration step as described above.

The anion chromatography may be performed by loading the pool from the cation exchange chromatography step on a column or membrane previously equilibrated in phosphate buffer at pH 6-8. Before loading, the material may be diluted to a conductivity of 2-12 mS/cm with water and pH adjusted to target pH. The product is collected in the flow-through fraction.

In one embodiment, a virus filtration is performed after the anion chromatography. The virus filtration may be performed as described above. In one embodiment, a virus filtration is performed both after the cation chromatography and after the anion chromatography.

In one embodiment, the anion chromatography is performed as flow-through using a membrane or a solid resin

Buffer change and concentration of the antibody may be performed following the last chromatography step, if desirable (see for instance Pete Gagnon: Purification Tools for monoclonal Antibodies (1996) ISBN-9653515-9-9) and for instance in WO2009010269.

The antibody sample may also be further formulated into a pharmaceutical preparation, as in a preparation that is suitable for pharmaceutical use, as it is known in the art.

The present invention also provides an antibody purification platform, that is, a standarized method, which is useful for producing a wide selection of different antibodies, a one-size-fits-all, which platform comprises a method according to the invention.

Such a standardized platform has several benefits in the production:
saves development/testing cost and time as the same process may be applied for each project
same equipment and raw materials, buffers etc. may be applied—thus, no need for extra testing and to get new suppliers approved etc.
virus validation studies may be reused from ne project to the next
saves man-power and secures product quality.

In one embodiment, such antibodies are IgG antibodies

An antibody production platform comprising a method of the present invention will have additional benefits, for instance less leakage of affinity ligand and the possibility of eluting different IgG subtypes under the same conditions.

Accordingly, the present invention also provides a process for the industrial production of antibodies, wherein said process comprises a method according to the present invention for purifying an antibody compound. In one embodiment, such antibody compounds are therapeutic antibodies. In one embodiment, such antibody compounds are IgG antibodies.

In one embodiment, the present invention provides a process for the industrial production of antibodies, wherein said process comprises a method according to the present invention for purifying an antibody compound, wherein the steps following the eluation from the Protein A derivative/analogue chromotography is performed in a continous process mode without holding tanks.

The following is a non-limiting list of embodiments of the present invention.

Embodiment 1: A method for purifying an antibody compound from a suspension comprising said antibody compound, wherein
i) said suspension is brought into contact with a Protein A derivative/analogue under conditions, wherein the Protein A derivative/analogue binds the antibody compound,
ii) said Protein A derivative/analogue bound antibody is washed with a suitable buffer, and
iii) said antibody compound is eluted from the Protein A derivative/analogue with a suitable buffer and collected in a resulting eluate.

Embodiment 2: A method according to embodiment 1, wherein said Protein A derivative/analogue is a Protein A derivative/analogue, wherein alkali-labile amino acids in the IgG binding domain is replaced with more alkali-stable amino acids.

Embodiment 3: A method according to embodiment 1 or embodiment 2, wherein said Protein A derivative/analogue is attached to an inert resin.

Embodiment 4: A method according to embodiment 3, wherein said Protein A derivative/analogue resin is MabSelect SuRe™.

Embodiment 5: A method according to any of embodiments 1 to 4, wherein one or more of steps i) to iii) is performed at a temperature below room temperature.

Embodiment 6: A method according to embodiment 5, wherein all steps i), ii) and iii) are performed at a temperature below room temperature.

Embodiment 7: A method according to embodiment 5 or embodiment 6, wherein the temperature below room temperature is a temperature of from 2 to 25° C., such as from 5 to 25° C., for instance from 10 to 25° C., such as from 15 to 25° C.

Embodiment 8: A method according to embodiment 5 or embodiment 6, wherein the temperature below room temperature is a temperature selected from a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C.

Embodiment 9: A method according to embodiment 5 or embodiment 6, wherein the temperature below room temperature is a temperature selected from a temperature of from 2 to 10° C., such as from 5 to 10° C.

Embodiment 10: A method according to embodiment 5 or embodiment 6, wherein the temperature below room temperature is a temperature of from 2 to 5° C.

Embodiment 11: A method according to any of embodiments 1 to 7, wherein the chromatography is performed in flow-through mode using a membrane or a solid resin.

Embodiment 12: A method for purifying an antibody compound from a suspension comprising said antibody compound, wherein
i) said suspension is brought into contact with ligand having affinity for such antibody under conditions, wherein said ligand binds the antibody compound,
ii) said ligand bound antibody is washed with a suitable buffer,
iii) said antibody compound is eluted from the ligand with a suitable buffer and collected in an eluate, and
   the eluate from step iii) is subjected to cation chromatography.

Embodiment 13: A method according to embodiment 12, wherein said ligand is Protein A.

Embodiment 14: A method according to any of embodiments 1 to 11, wherein the eluate from step iii) is subjected to cation chromatography.

Embodiment 15: A method according to any of embodiments 12 to 14, wherein the eluate from step iii) is subjected to virus inactivation prior to being subjected to the cation chromatography.

Embodiment 16: A method according to embodiment 15, wherein the pH of the eluate from step iii) is lowered to a pH of from 3 to 4 for a period of from 5 minutes to a day and then readjusted prior to the cation chromatography.

Embodiment 17: A method according to embodiment 16, wherein the pH is lowered to a pH of from 3.4-3.9 for a period of from 20 to 90 minutes.

Embodiment 18: A method according to any of embodiments 12 to 17, wherein the cation chromatography is performed at a temperature below room temperature.

Embodiment 19: A method according to embodiment 18, wherein the temperature below room temperature is a temperature of from 2 to 25° C., such as from 5 to 25° C., for instance from 10 to 25° C., such as from 15 to 25° C.

Embodiment 20: A method according to embodiment 18 or embodiment 19, wherein the temperature below room temperature is a temperature selected from a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C.

Embodiment 21: A method according to embodiment 18 or embodiment 19 wherein the temperature below room temperature is a temperature selected from a temperature of from 2 to 10° C., such as from 5 to 10° C.

Embodiment 22: A method according to embodiment 18 or embodiment 19, wherein the temperature below room temperature is a temperature of from 2 to 5° C.

Embodiment 23: A method according to any of embodiments 12 to 22, wherein said cation chromatography is performed as flow-through using a membrane or a solid resin.

Embodiment 24: A method according to any of embodiments 1 to 7, wherein the eluate from step iii) is subjected to anion chromatography.

Embodiment 25: A method according to embodiment 24, wherein the conductivity of the eluate from step iii) is adjusted to a conductivity of from 2 to 12 mS/cm before loading.

Embodiment 26: A method according to embodiment 24 or embodiment 25, wherein the eluate from step iii) is subjected to virus inactivation prior to being subjected to the anion chromatography.

Embodiment 27: A method according to embodiment 26, wherein the pH of the eluate from step iii) is lowered to a pH of from 3 to 4 for a period of from 5 minutes to a day and then readjusted prior to the anion chromatography.

Embodiment 28: A method according to embodiment 27, wherein the pH is lowered to a pH of from 3.4-3.9 for a period of from 20 to 90 minutes.

Embodiment 29: A method according to any of embodiments 12 to 23, wherein the eluate from the cation chromatography is subjected to anion chromatography, wherein the eluate from the cation chromatography is optionally subjected to a virus filtration prior to being subjected to anion chromatography.

Embodiment 30: A method according to embodiment 29, wherein the eluate from the cation chromatography is subjected to a virus filtration prior to being subjected to anion chromatography.

Embodiment 31: A method according to embodiment 30, wherein the virus filtration comprises a filtration of the eluate from the cation chromatography on a first and then on a second filter.

Embodiment 32: A method according to embodiment 42, wherein the first filter is more porous than the second filter.

Embodiment 33: A method according to embodiment 29, wherein the conductivity of the eluate from the cation chromatography is adjusted to a conductivity of from 2 to 12 mS/cm before loading.

Embodiment 34: A method according to any of embodiments 24 to 33, wherein the anion chromatography is performed at a temperature below room temperature.

Embodiment 35: A method according to embodiment 34, wherein the temperature below room temperature is a temperature of from 2 to 25° C., such as from 5 to 25° C., for instance from 10 to 25° C., such as from 15 to 25° C.

Embodiment 36: A method according to embodiment 34 or embodiment 35, wherein the temperature below room temperature is a temperature selected from a temperature of from 2 to 20° C., such as from 5 to 20° C., for instance from 10 to 20° C., such as from 15 to 20° C., or at a temperature of from 2 to 15° C., such as from 5 to 15° C., for instance from 10 to 15° C.

Embodiment 37: A method according to embodiment 34 or embodiment 35, wherein the temperature below room temperature is a temperature selected from a temperature of from 2 to 10° C., such as from 5 to 10° C.

Embodiment 38: A method according to embodiment 34 or embodiment 35, wherein the temperature below room temperature is a temperature of from 2 to 5° C.

Embodiment 39: A method according to any of embodiments 24 to 38, wherein said anion chromatography is performed as flow-through using a membrane or a solid resin.

Embodiment 40: A method according to any of embodiments 24 to 39, wherein the eluate from the anion chromatography is subjected to a virus filtration.

Embodiment 41: A method according to embodiment 40, wherein the virus filtration comprises a filtration of the eluate from the anion chromatography on a first and then on a second filter.

Embodiment 42: A method according to embodiment 42, wherein the first filter is more porous than the second filter.

Embodiment 43: A method according to any of embodiments 1 to 42, wherein the final eluate is subjected to diafiltration and/or ultrafiltration.

Embodiment 44: A method according to any of embodiments 1 to 43, wherein the final eluate is formulated into a pharmaceutical preparation.

Embodiment 45: A method according to any of embodiments 1 to 44, wherein the antibody compound is an IgG antibody.

Embodiment 46: A method according to any of embodiments 1 to 45, wherein the antibody compound is a therapeutic antibody.

Embodiment 47: An antibody purifying platform, which platform comprises a method according to any of embodiments 1 to 46.

Embodiment 48: An antibody purifying platform suitable for purifying IgG antibodies, which platform comprises a method according to any of embodiments 1 to 46.

Embodiment 49: An antibody purifying platform according to embodiment 47 or embodiment 48, wherein said platform is used for production of IgG antibodies.

Embodiment 50: An antibody purifying platform according to any of embodiments 47 to 49, wherein said platform is used for production of therapeutic antibodies.

Embodiment 51: A process for the industrial production of antibodies, wherein said process comprises a method according to any of embodiments 1 to 46.

Embodiment 52: A process according to embodiment 51, wherein the steps following the eluation from the Protein A derivative/analogue chromotography is performed in a continous process mode without holding tanks.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (for instance all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (“for instance”, “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (for instance a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

### EXAMPLES

### Example 1

### Antibody Purification Process

The purification process of the mAbs (anti-NKG2A as described in for instance WO2006070286 and WO2008009545, anti-NKG2D, as described for instance in WO2005097160 and anti-C5aR, described for instance in WO2003062278 and WO2008022390) from the CHO cell culture comprise several steps. The cell culture supernatant was filtrated and loaded on a 106 ml MabSelect SuRe affinity column (length 11 cm) (for solvents and conditions, see below). The eluted product pool was subjected to virus inactivation by adjusting pH to pH 3.7 with 0.2 M citric acid and kept at room temperature for 1 hour. Elution may also be performed using other buffers with low pKa such as citric or acetic acid in a concentration of 5-100 mM. Subsequently, the pool was adjusted to pH 5.0 with 0.5 M Na₂HPO₄ and then it was loaded on a 94 ml POROS 50 HS cation exchange column (length 4.8 cm). After pH adjustment, precipitation of impurities in the solution may take place and this have to be removed by filtration or centrifugation before loading on the cation column. Elution was achieved with an elution buffer of 25 mM CH₃COONa, pH 5.0 with a 0-0.3 mol/kg NaCl gradient over 10 CV. pH for this step may be adjusted according to pl of the actual mAb. The mAb pool was adjusted to pH 7.0 with a 0.5 M Na₂HPO₄ solution and filtrated through a filter train consisting of a0.1 μm filter (4.52 cm²) followed by a Planova 20 N virus filter (0.001 m²). (An alternative to the Millex-VV filter is a 0.1 μm Millipore Opticap XLT 20 filter). The virus filtrate was diluted with water to ≦7.0 mS/cm at room temperature before loading it on a Sartobind Q SingleSep capsule (75 cm²) anion-exchange membrane. Reduction of the conductivity may also be achieved by UF/DF. The anion-exchange membrane step was run in flow-through mode at non-binding conditions and the filtrate was finally ultra- and diafiltrated with a 50 cm² Biomax 30k membrane on an Äkta cross-flow equipment into a 10 mM Histidine buffer pH 6.2, followed by addition of Tween 80 to 0.01%. The end composition of the drug substance formulation was 40 mg mAb/mL, 25 g/L sucrose, 0.01% w/w Tween 80, 10 mM Histidine, pH 6.2. Step yield and other conditions/results for Anti-NKG2A, Anti-NKG2D and Anti-05aR are given in Table 1, 2 and 3, respectively. Conductivity and pH of the Q membrane step and the loading solution passed through the membrane will have to be adjusted for each mAb to achieve maximum reduction of impurities with highest possible yield. The conductivity and pH may thus vary in the range of 2-12 mS/cm (controlled by the NaCl content) and pH 5.8-8.0, respectively.

Solvents and Conditions:

Protein A Derivative/Analogue Capture—MabSelect SuRe
Room temperature
Flow rate: 20 column volumes per hour (CV/h)
Load: 30 g mAb/L resin, however, it may be varied in the range of 1-50 g/L resin
   Equilibration and washing buffer: 11.5 mmol/kg NaH₂PO₄, 38.5 mmol/kg Na₂HPO₄, 300 mmol/kg NaCl
   Washing buffer: 6.5 mmol/kg NaH₂PO₄, 43.5 mmol/kg Na₂HPO₄, 1000 mmol/kg NaCl
   Elution buffer: 10 mmol/kg formic acid, pH 3.5
   Eluting using a step gradient
Virus inactivation at pH 3.7 (0.2 M citric acid) for 1 hour followed by pH adjustment to pH 5.0 with 0.5 M Na₂HPO₄

CIEX—Poros 50 HS, pH 5
Room temperature
Flow rate: 25 CV/h
Load: 45 g mAb/L resin, however, it may be varied in the range of 40-120 g/L resin
   Equilibration and washing buffer: 25 mmol/kg CH₃COONa and 12.5 mmol/kg CH₃COOH
   Elution buffer: 25 mmol/kg CH₃COONa and 10.1 mmol/kg CH₃COOH, 300 mmol/kg NaCl
   Eluting using linear gradient from 0-300 mmol/kg NaCl in equilibration/elution buffer. buffer, pH 5

Virus Filtration—Planova 20N
Room temperature
Pressure during filtration 0.8 bar
Load of: ≦110 kg/m², however, it may be up to 500 kg/m²
   Adjustment pH to 7.0 with a 0.5 M Na₂HPO₄ solution
   Equilibration and washing buffer: 7.7 mmol/kg NaH₂PO₄, 12.2 mmol/kg Na₂HPO₄, 50 mmol/kg NaCl
   Prefiltration with a 0.1 μm filter
   Filtrate with Planova 20N

Q Membrane Flow-Through, pH 7
Room temperature
Flow rate: 300 CV/h
Load: 483 g/m², however, the load may be varied in the range of 200-3000 g/m²
   Dilution of pool to 7 mS/cm with water
   Equilibration and washing buffer: 7.7 mmol/kg NaH₂PO₄, 12.2 mmol/kg Na₂HPO₄, 50 mmol/kg NaCl
   Application and collection of flow-through

UF/DF—30 kDa Biomax
Buffer change into 10 mmol/kg Histidine, pH 6.2-6.5
Concentration and formulation to 30-60 mg/mL in 25 g/L glucose and 0.01% Tween80, pH 6.2-6.5

TABLE 1
Summary of Anti-NKG2A purification

| | | Conductivity | Content | Yield | HMWP | HCP |
|---|---|---|---|---|---|---|
| Process step | pH | (ms/cm) | (mg/ml) | (%) | (%) | (ppm) |
| Cell culture | 7.0 | — | 2.6 | — | — | 540000 |
| Affinity pool | 6.4 | 4.1 | — | — | — | — |
| Affinity pool II | 5.0 | — | 9.0 | 92 | 0.90 | 849 |
| CIEC load | 5.1 | — | 8.9 | — | 0.96 | 264 |
| CIEC pool | 4.8 | 18.5 | 6.1 | 86 | 0.99 | 60 |
| AIE membrane | 7.1 | 7.0 | 1.8 | — | 0.91 | 63 |
| load | | | | | | |
| Q membrane | 7.1 | 7.6 | 1.8 | 102 | 0.87 | 51 |
| pool | | | | | | |
| UF/DF load | 7.0 | 7.4 | 1.7 | — | — | — |
| UF/DF filtrate | 6.2 | — | 40.0 | 81 | 1.18 | 45 |

Description of analytical methods are given in Example 9.

TABLE 2
Summary of Anti-NKG2D purification

| | | | | | | Protein A |
|---|---|---|---|---|---|---|
| | | Content | Yield | HMWP | HCP | leakage |
| Process step | pH | mg/ml | (%) | (%) | (ppm) | (ppm) |
| Cell culture | 7.0 | 3.5 | — | — | — | |
| Affinity pool | — | 11.8 | — | — | — | — |
| CIEC pool | 5.0 | 11.8 | 96 | 1.0 | 286 | 0.09 |
| Q membrane | 7.0 | 2.8 | 98 | 0.90 | 10 | — |
| pool | | | | | | |
| UF/DF filtrate | 6.2 | 38.2 | 91 | 0.90 | 4.6 | 0.08 |

Description of analytical methods are given in Example 9.

TABLE 3
Summary of Anti-C5aR purification

| | | | | | | Protein A | Purity |
|---|---|---|---|---|---|---|---|
| | | Content | DNA | HMWP | HCP | leakage | (%) |
| Process step | pH | mg/ml | (ppm) | (%) | (ppm) | (ppm) | SDS-PAGE |
| Cell culture | 7.0 | 1.2 | — | — | 515000 | | |
| Affinity pool | — | 9.6 | 760 | 1.3 | 950 | 1.7 | 98.8 |
| CIEC pool | 5.0 | 7.8 | <50 | 0.8 | 22 | 0.3 | 99.4 |
| Q membrane | 7.0 | 2.0 | — | 1.0 | <18 | — | 99.4 |
| pool | | | | | | | |
| UF/DF filtrate | 6.2 | 40.1 | 0.02 | 1.6 | 2.5 | 0.3 | 99.6 |

Description of analytical methods are given in Example 9.

### Example 2

### Capture of Anti-Interferon-Alpha (Anti-IFNα) on MabSelect SURE

Cell culture supernatant from a CHO cell culture was filtered on a 0.45 μm filter using a 1.2 μm filter as pre-filter. The titer of anti-IFNα (as described in for instance WO2006086586) produced by the cells was 2 mg/ml.

The pl of the monoclonal antibody was 7.6. A MabSelect SURE column (56 ml volume, height 10.5 cm, diameter 2.6 cm) was previously equilibrated with 10 column volumes (CV) of 50 mM sodium phosphate, 300 mM NaCl, pH 7.0; flow rate was at 20 CV/h. The column was loaded with 840 ml filtrated cull culture supernatant operated at a flow rate of 20 CV/h; loading capacity was about 30 mg/ml matrix material.

Before elution, the column was washed with 10 CV of 50 mM sodium phosphate+300 mM NaCl, pH 7.0 followed by a wash with 6 CV of 50 mM sodium phosphate+1000 mM NaCl, pH 7.0 and a 5 CV wash with 50 mM sodium phosphate+300 mM NaCl, pH 7.0.

Elution was achieved with elution buffer made up of 10 mM formic acid, pH 3.5. Immediately after elution, fractions of eluate comprising the antibody pool were pH adjusted to pH 3.7 using a 0.2 M citric acid solution and held at pH 3.7 for 1 h. Next, the solution was adjusted to pH 5.0 using 0.5 M Na₂HPO₄.

The antibody concentration and was determined as described above. The recovery based on the titer of the cell culture supernatant solution prior to loading was 78%; the concentration of antibody in eluate solution was 7 mg/ml.

### Example 3

Capture of Anti-Factor IX (Anti-FIX) on MabSelect SURE

Cell culture supernatant from a CHO cell culture was filtered on a Sartobran P 10″ 0.65 μm+0.45 μm filter. The titer of anti-FIX produced by the cells was 2 mg/ml.

The pl of the monoclonal antibody was 7.5. A MabSelect SURE column (2.4 l volume) was previously equilibrated with 10 column volumes (CV) of 50 mM sodium phosphate, 300 mM NaCl, pH 7.0; flow rate was at 12.5 CV/h. The column was loaded with 9 L filtered cull culture supernatant operated at a flow rate of 30 CV/h; loading capacity was about 7.5 g/l matrix material.

Before elution, the column was washed with 2 CV of 50 mM sodium phosphate, 300 mM NaCl, pH 7.0 followed by a wash with 6 CV of 50 mM sodium phosphate, 1000 mM NaCl, pH 7.0 and a 5 CV wash with 50 mM sodium phosphate, 300 mM NaCl, pH 7.0.

Elution was achieved with elution buffer made up of 10 mM formic acid, pH 3.5. Immediately after elution, fractions of eluate comprising the antibody peak were pH adjusted to pH 3.7 using a 0.2 M citric acid solution and held at pH 3.7 for 1 h. Next the solution was pH adjusted to pH 5.0 using 0.5 M Na₂HPO₄.

The antibody concentration in the eluate pool was determined by measuring absorbance at 280 nm using an extension coefficient of 1.71 cm-1. The antibody concentration in the culture supernatant was determined by the SE-HPLC method used for determination of monomeric IgG content and % HMWP by comparing the area of the anti-FIX monomeric peak with a reference sample with known concentration.

The recovery of anti-FIX based on the titer of the cell culture supernatant solution prior to loading was around 100%; the concentration of antibody in eluate solution was 5 g/l.

### Example 4

CIEX of Anti Interferon Alpha (Anti-IFNα) on POROS 50 HS

1519 ml of anti-IFNα, previously purified on a MabSelect SURE column and pH adjusted as described in Example 2, in a concentration of 2.6 mg/ml was filtered on a HVLP type 0.45 μm filter from Sartorius. The filtered antibody solution was loaded onto a POROS 50 HS column (94 ml volume, height 4.8 cm, diameter 5.0 cm) that previously was equilibrated with 10 column volumes (CV) of 25 mM Na-Acetate, 12.4 mM acetic acid, pH 5.0; flow rate was 25 CV/h; loading capacity was about 43 mg/ml matrix material.

Before elution, the column was washed with 10 CV of 25 mM Na-Acetate, 12.4 mM acetic acid.

Elution was achieved with a linear gradient of elution buffer over 10 CV made up of 25 mM Na-Acetat, 10.1 mM acetic acid, 300 mM NaCl, pH 5.0. An anti-IFNα containing solution was achieved by collecting from OD 0.250 at the leading edge to OD1.125 on the trailing edge.

The antibody concentration in the eluate pool and in the loading solution was determined by measuring absorbance at 280 nm in the samples using an extension coefficient of 1.63 cm-1·(g/L)-1. The recovery of anti-IFNα was 72%; the concentration of anti-IFNα in eluate solution was 5.4 mg/ml.

Over the CIEX purification step the amount of high molecular weight proteins (using the SE-HPLC method described above) in the process fluid was reduced from 14.5% to 2%.

### Example 5

### CIEX of Anti Factor IX (Anti-FIX) on POROS 50 HS

490 ml of anti-FIX (previously purified on a MabSelect SURE column and pH adjusted as described in Example 3) in a sodium phosphate buffered solution at pH 5.0 at a protein concentration of 2.7 mg/ml was loaded onto a POROS 50 HS column (45 ml volume, height 8.5 cm, diameter 2.6 cm) that previously was equilibrated with 5 column volumes (CV) of 25 mM Na-Acetate, 12.4 mM acetic acid, pH 5.0; flow rate was 25 CV/h; loading capacity was about 43 mg/ml matrix material.

Before elution, the column was washed with 10 CV of 25 mM Na-Acetate, 12.4 mM acetic acid.

Elution was achieved with a linear gradient of elution buffer over 10 CV made up of 25 mM Na-Acetate, 10.1 mM acetic acid, 300 mM NaCl, pH 5.0. An anti-FIX containing solution was achieved by collecting from OD 0.20 at the leading edge to OD 0.4 on the trailing edge.

The antibody concentration in the eluate pool and in the loading solution was determined by measuring absorbance at 280 nm in the samples using an extension coefficient of 1.71 cm-1·(g/L)-1. The recovery of anti-FIX was 91%; the concentration of antibody in eluate solution was 3.6 mg/ml.

### Example 6

### Flow Through AIEX of Anti Factor IX (Anti-FIX) on Sartobind Q SingleSep Nano Capsule Q-Membrane

180 ml of anti-FIX, previously purified as described in Example 5, contained in a sodium acetate solution at pH 5.1 and a conductivity of 15.7 mS/cm was adjusted to pH 7.0 with 0.5 M di-sodium phosphate (temperature 15.7° C.). Conductivity was adjusted to 7.00 mS/cm with water. After conductivity adjustment pH was 7.03 (temperature 20.1° C.). Volume of the pH and conductivity adjusted sample was 540 ml and antibody concentration was 1.15 mg/ml.

500 ml of the solution was passed through a SingleSep nano capsule Q-membrane (membrane volume 1 ml) that previously was equilibrated with 15 membrane volumes (MV) of 20 mM sodium phosphate, 50 mM NaCl, pH 7.0; flow rate was 10 MV/h. The membrane was subsequently washed with 10 MV of 20 mM Na-phosphate, 50 mM NaCl, pH 7.0.

The antibody concentration in the collected flow-through pool and in the loading solution was determined by measuring absorbance at 280 nm in the samples using an extension coefficient of 1.71 cm-1·(g/L)-1. The recovery of anti-FIX was 95% and concentration of antibody in eluate solution was 1.1 mg/ml.

### Example 7

### Ultrafiltration/Dia Filtration of Anti Interferon Alpha (Anti-IFNα) on Biomax 30K Ultra Filtration Filter

520 ml of anti-IFNα at a concentration of 5.7 mg/ml contained in a Na-acetate solution pH 5.0 and approximately 0.2 M NaCl was up concentrated to 45 ml and a antibody concentration of 51 mg/ml on a Biomax 30K Pellicon XL filter previously equilibrated with 34 mM Histidine, pH 6.5. The concentrated sample was buffer exchanged on the Biomax 30K Pellicon XL filter 6 times with 50 ml of 34 mM Histidine, pH 6.5.77% of the antibody was recovered after ultra filtration and diafiltration.

14 ml of the buffer exchanged anti-IFNα concentrate was added sucrose to a final concentration of 86 mg/ml and tween 80 to a final concentration of 0.03%. Finally the solution was filtered through a 0.22 μm filter

Concentration of anti-IFNα was determined by measuring absorbance at 280 nm in the samples using an extension coefficient of 1.63 cm⁻¹ (g/L)⁻¹.

### Example 8

### Purification of mAb on MabSelect SuRe in Cold Room

An antibody, Anti-IL20, was purified using MabSelect SuRe resin for capture. The experiment was performed at cold room temperature. The cold room temperature experiment was compared with an identical experiment performed at room temperature. Conditions were as follows:

Cell culture supernatant from a CHO cell culture (2.6 g mAb/l) was filtrated and loaded on the MabSelect SuRe column after the column had been equilibrated with 10 CV of 11.5 mmol/kg NaH₂PO₄+38.5 mmol/kg Na₂HPO₄+300 mM NaCl, pH 7.0. A 5 ml column was operated with a flow rate of 20 CV/hour, and the column was washed with 10 CV equilibration buffer, followed by 10 CV of 6.5 mmol/kg NaH₂PO₄+43.5 mmol/kg Na₂HPO₄+1000 mM NaCl, pH 7.0, followed by 10 CV of equilibration buffer before elution. Elution was achieved with an elution buffer of 10 mM formic acid, pH 3.5 and after elution the pool including the mAb was pH adjusted to pH 3.7 with 0.2 M citric acid and subsequently adjusted to pH 5.0 with 0.5 M Na₂HPO₄. Levels of Protein A derivative/analogue leakage and aggregates are shown in Table 4.

TABLE 4

| | Protein A derivative/analogue | Aggregates |
|---|---|---|
| | leakage (ppm) | (%) |
| MabSelect SuRe pool - cold | 0.3 | 0.86 |
| MabSelect SuRe pool - | 3.4 | 1.68 |
| room temperature | | |

Conclusion: From Table 2 it can be seen that purifying an antibody on an affinity column based on a Protein A derivative/analogue at cold room temperature provides a significant reduction in the Protein A derivative/analogue leakage (˜10 fold) and HMWP (aggregate) levels in the pool (˜2 fold), compared to purifying the same antibody at room temperature.

### Example 9

### Analytical Methods

### Determination of Anti Body Content by Protein A HPLC

The content of antibody was determined using a Protein A HPLC method. The samples were analysed using an ImmunoDetection Cartridge Protein A column (diameter 2.1 mm, length 3 mm). With a flowrate of 1 ml/min the column is equilibrated for 3 minutes with a 25 mM sodium phosphate, 0.5 M NaCl, pH 7.5. The column is loaded with approximately 30 μg of anti body. The column is washed with the equilibration buffer and finally eluted for 2 minutes at 1 ml/min with buffer containing 10 mM HCOONa, pH 3.5.

Content of anti body is determined by comparing the area under the eluted main peak with a reference sample with known anti body concentration.

### Determination of Monomeric IgG Content and % High Molecular Weight Proteins (HMWP)

The purity by HPLC is determined using a size exclusion chromatography (SE-HPLC) method. The samples are analysed using a TSK G3000 SWXL column (diameter 7.8 mm, length 30 mm), isocratic elution (elution buffer 200 mM Sodium phosphate, 300 mM NaCl, 10% 2-propanol and pH 6.9) and subsequent UV detection at 280 nm. This method is used to determine monomeric IgG content (retention time approximately 9.5 minutes) and % HMWP (retention time 7-8.5 min.) consisting of dimeric species or larger which are separated according to size by the gel resin. The monomeric content and % HMWP are determined relative to the total protein content detected by the method.

### Determination of CHO Host Cell Protein

The CHO host cell protein is determined by a two step sandwich ELISA method. A measurement involves the capture of any host cell protein present in a sample with polyclonal rabbit HCP antibodies immobilised on a microtitre plate. The bound HCP is detected by subsequent addition of a polyclonal rabbit HCP antibody conjugated to biotin, which in turn is detected by avidin conjugated to horseraddish peroxidase. Quantification is based on incubation with the chromogenic substrate 3,3″,5,5″-tetramethylbenzidine (TMB). The microtitre plate is read at 450 nm (with a reference wawelength of 620 nm).

### Determination of Protein A Derivative Leakage

Protein A derivative leakage is determined by a two step sandwich ELISA method, using a commercially available kit. A measurement of Protein A derivative in the product involves the capture of Protein A derivative present in a sample with polyclonal chicken anti-Protein A antibodies immobilised on a microtitre plate. The Protein A derivative is detected by subsequent addition of polyclonal rabbit anti-Protein A antibodies conjugated to biotin, which in turn is detected by avidin conjugated to horseraddish peroxidase. Quantification is based on incubation with the cromogenic substrate TMB. The microtitre plate is read at 450 nm (with a reference wawelength of 620 nm).

### Example 10

### CIEX of Antibody (Anti-KIR) on POROS 50 HS at 4° C.

Chromatography system (Äkta Explorer100) and solvents were placed in a refrigerator set to 4° C.

20.3 mL of antibody (Anti-KIR as described in for instance WO2005003168, WO2005003172 or WO2006003179) in a concentration of 6.2 mg/mL was loaded onto a POROS 50 HS column (3.1 mL volume, 1 cm diameter, 4 cm length) that previously was equilibrated with 10 column volumes (CV) of 25 mM Na-Acetate, 12.4 mM acetic acid, pH 5.0; flow rate was 25 CV/h.

Before elution, the column was washed with 10 CV of 25 mM Na-Acetate, 12.4 mM acetic acid.

Elution was achieved with a linear gradient of elution buffer over 10 CV made up of 25 mM Na-Acetat, 10.1 mM acetic acid, 300 mM NaCl, pH 5.0. An antibody (Anti-KIR) containing solution was achieved by collecting from OD 1.0 at the leading edge to OD 1.0 on the trailing edge. The column was regenerated by 5 CV of 1 M NaOH followed by 5 CV of 2 M NaCl, 50 mM acetic acid and 10 CV of 25 mM Na-Acetate, 12.4 mM acetic acid, pH 5.0.

The antibody concentration in the eluate pool and was determined from the chromatogram (absorbance at 280 nm and extinction coefficient=1.49 (g/L)-1·cm-1). The recovery of the antibody based on the concentration in the loading solution was 97%; the concentration of antibody in eluate solution was 3.7 mg/mL.

Over the CIEX purification step, the amount of HCP (host cell protein) in the process fluid was reduced a factor 3. The yield of a reference experiment (exact same method and starting material) at room temperature (20° C.) was 92%. CIEX at low temperature could preferably be used for antibodies being instable at room temperature.

### Example 11

### CIEX of anti-IL20 on POROS 50 HS with Very High Loading

### Flow-Through Mode

Chromatography system (Äkta Explorer100) and solvents placed at 20° C. 215 mL of antibody (anti-IL20 for instance as described in WO9927103) in a concentration of 10 mg/mL was loaded onto a POROS 50 HS column (3.1 mL volume) that previously was equilibrated with 10 column volumes (CV) of 25 mM Na-Acetate, 12.4 mM acetic acid, pH 5.0; flow rate was 25 CV/h. The column was after load washed with 5 CV of 25 mM Na-Acetate, 12.4 mM acetic acid.

The antibody concentration in the collected flow-through pool and was determined from the chromatogram (absorbance at 280 nm and extinction coefficient=1.52 (g/L)-1·cm-1). The recovery of the antibody based on the concentration in the loading solution was 91%; the concentration of antibody in collected pool was about 9 mg/mL. Over the CIEX purification step the amount of HCP (host cell protein) in the process fluid was reduced a factor of 7. To obtain maximum reduction of impurities, an adjustment of pH for the chromatographic process may be necessary in the range of pH 4.5-6.0.

The collected pool can be further processed on the flow through AIEX as described in Example 6.

### Example 12

### Capture of Antibody Anti-IFNα on MabSelect SuRe

Cell culture supernatant from a CHO cell culture was crudely purified by filtration (Clarigard 3.0 μm, Polysep 1/0.5 μm, Durapore 0.22 μm). The titer of the antibody produced by the cells was 3.4 mg/ml.

The pl of the monoclonal antibody was 7.7. A MabSelect SuRe column (1000 ml volume, 13 cm height, 10 cm diameter) was previously equilibrated with 5 column volumes (CV) of 20 mM phosphate (Na₂HPO₄/NaH₂PO₄), 150 mM NaCl, pH 7.2; flow rate was at 24 CV/h. The column was loaded with 10750 ml filtrated cull culture supernatant operated at a flow rate of 18 CV/h; loading capacity was about 36 mg/ml matrix material.

Before elution, the column was washed with 4 CV of 20 mM phosphate (Na₂HPO₄/NaH₂PO₄), 150 mM NaCl, pH 7.2. Elution was achieved with 10 CV elution buffer made up of 10 mM formic acid, pH 3.5 with a flow rate of 6 CV/h. Immediately after elution, fractions of eluate comprising the antibody pool were pH adjusted from pH 4.0 (conductivity 0.12 mS/cm) to pH 3.7 using a 0.2 M citric acid solution and held at pH 3.7 for 1 h at room-temperature. Next, the solution was adjusted to pH 6.1 using 0.5 M Na₂HPO₄. The material was then filtered prior to storage (0.8+0.45 μm, Sartopore 2 300, 0.03 m²).

Antibody yield from this step was 62% and the concentration of antibody in eluate solution was 9.1 mg/ml.

### Example 13

### Capture of Antibody Anti-IL-20 on MabSelect SuRe

Cell culture supernatant from a transiently transfected culture was crudely purified by filtration. The pl of the monoclonal antibody was 7.1. A MabSelect SuRe column (1 ml volume, height 2.5 cm, diameter 0.7 cm) was previously equilibrated with 10 column volumes (CV) of 20 mM phosphate (Na₂HPO₄/NaH₂PO₄), 150 mM NaCl, pH 7.2; flow rate was at 60 CV/h. The column was loaded with 500 ml filtrated cull culture supernatant operated at a flow rate of 30-60 CV/h.

Before elution, the column was washed with 25 CV of 20 mM phosphate (Na₂HPO₄/NaH₂PO₄), 150 mM NaCl, pH 7.2. Elution was achieved with 20 CV elution buffer in a linear gradient from 0 to 100%. The elution buffers tested were made up of (1) 10 mM citric acid pH 3.0, (2) 0.1 M glycine pH 3.0 or (3) 10 mM formic acid pH 3.5. The elution was performed with a flow rate of 60 CV/h. The column was regenerated by additional 10 CV elution buffer ((1) 10 mM citric acid pH 3.0, (2) 0.1 M glycine pH 3.0 or (3) 10 mM formic acid pH 3.5) and then 5 CV 0.1 M NaOH. The column was re-equilibrated with 10 CV of 20 mM phosphate (Na₂HPO₄/NaH₂PO₄), 150 mM NaCl, pH 7.2. Yields were in the range of 85-90%.

### Example 14

### Fab₂ Purification Process

The purification process of the Fab₂ fragment of Anti-KIR from the CHO cell culture consists of the following steps: Affinity capture, Virus inactivation/cleavage (pepsination), and cation-exchange chromatography. Purification was performed as described below.

### Overall Process:

The cell culture supernatant from a CHO cell culture was filtrated and loaded on a 500 ml MabSelect SuRe affinity column (for solvents and conditions, see below). Elution was achieved with an elution buffer of 60 mM Na-citrate pH 4.0 and after elution the mAb pool was adjusted to pH 3.75 with cold 0.5 M HCl. 10 mg pepsin/g mAb was added and incubated at 37° C. for 3 to 6 hours. Subsequently the pool was adjusted to pH 7.0 by addition of cold 0.5 M NaOH and incubated for at least 8 hours at 4° C. After incubation pH of the pool was adjusted to 5.0. The pool was furthermore diluted with H₂O to a conductivity below 2 mS/cm and loaded on 500 ml SOURCE 30S in a FineLINE 100 column. Elution was achieved with a linear gradient of 0-0.2 M NaCl over 20 CV in 20 mM NaOAc pH 5.0 buffer.

### Solvents and Conditions:

Affinity Chromatography:

Load: Cell supernatant is filtered through a 0.45 μm filter

Measure pH and conductivity.

Column material: MabSelect SuRe, 500 ml column XK50.

Buffer A: 20 mM Na-phosphate pH 7.2+150 mM NaCl

Buffer B: 60 mM Na-citrate pH 4.0

Buffer D: 0.1M NaOH

Cycle:
Regeneration with 3 CV Buffer B
Equilibration with 10 CV Buffer A
Wash with 10 CV Buffer A (extensive washing will remove some endotoxins)
Step elution with 15 CV Buffer B
CIP with 5 CV Buffer D
Re-equilibrate with Buffer A

Flow: 30-180 CV/h

Temperature of chromatography: Room temperature

Cleavage (Pepsination)

Pepsin from porcine gastric mucosa lyophilized powder in the amount of 3,200-4,500 units/mg protein from Sigma-Aldrich was used for cleavage.

Preparation of pepsin stock solution: Dissolve pepsin in H₂O at a concentration of 10 mg/ml.

Storage of the pepsin solution was done at −20° C.

mAb sample: Adjust pool from affinity step to pH 3.75 with cold 0.5 M HCl

Pepsination: Add 10 mg pepsin/g mAb to the sample, and mix and incubate at 37° C. for 3 to 6 hours. The reaction is controlled by SEC-HPLC. The reaction is stopped by addition of cold 0.5 M NaOH to pH 7 followed by incubation for at least 8 hours at 4° C. (over night).

Cation-Exchange Chromatography

Load solution preparation: Resulting solution from pepsination step is diluted with H₂O to a conductivity below 2 mS/cm. pH is then adjusted to 5.

Column material: SOURCE 30S, Column size 500 ml

Capacity at least 10 mg/ml column material

Buffer A: 20 mM NaOAc pH 5.0

Buffer B: 20 mM NaOAc pH 5.0+1.0 M NaCl

Stock solution 1: 100 mM EDTA+100 mM BenzamidineHCl

Cycle:
Equilibration with 10 CV buffer A
Loading of sample
Wash with 10 CV buffer A
First elution with salt gradient; 0-20% buffer B over 20 CV
Final elution with 5 CV of 100% buffer B
Regeneration with 1 M NaOH

Flow: 100 ml/min during fraction collection

Temperature of chromatography: Room temperature

### Example 15

### Affinity Chromatography Purification of Murine Anti-C5aR and Humanized Anti-C5aR

Affinity purification of the mAb from the CHO cell culture was performed as follows. The cell culture supernatant from a CHO cell culture was filtrated and loaded on a 1 ml MabSelect SuRe affinity column (for solvents and conditions, see below). Elution was achieved with an elution buffer of 10 mM formic acid, pH 3.0 or 10 mM citric acid, pH 3.0; and after elution the mAb pool was adjusted to pH 7.2 with 0.5 M NaH₂PO4, pH 7.6.

### Solvents and Conditions:

Column material: MabSelect SuRe (GE HealthCare cat no 17-5438-01), 1 ml column, 5 cm height×0.5 cm diameter

Buffer A: 20 mM Na-phosphate pH 7.2+150 mM NaCl

Buffer B1: 5 mM di-natriumhydrogencitrat pH 3

Buffer B2: 10 mM Sodium formate, pH 3.0

Buffer D: 0.1M NaOH

Buffer E: 0.5 M NaH₂PO4, pH 7.6

Cycle:
Regeneration with 3 CV Buffer B1 or B2
   Equilibration with 10 CV Buffer A
Loading of sample
   Wash with 10 CV Buffer A
Step elution with 15 CV Buffer B1 or B2
CIP with 5 CV Buffer D
Re-equilibrate with Buffer A

Fractions collection: Product fractions were pooled and pH was adjusted to 7.2 with buffer E

Flow rate: 30-180 cv/h

Temperature of chromatography: Room temperature

Description of analytical methods are given in Example 9.

### Example 16

### Formulation

The UF/DF step described in Example 1, was applied for Anti-KIR by buffer exchange into the following solutions:

50 mM phosphat, 250 mM Sucrose, 0.001% Tween 80, pH 7.

20 mM phosphat, 220 mM Sucrose, 0.001% Tween 80, pH 7.

The final concentration of antibody was 10 mg/ml in both solutions.

### Example 17

### CIEX with Very High Loading (Flow-Through Mode) Followed by Flow-Through AIEX

The chromatography system (Äkta Explorer100) and solvents were placed at 20° C.

478 mL of antibody (Anti-NKG2A) solution (affinity chromatography capture) at a concentration of 5.2 mg/mL was loaded onto a POROS 50 HS column (3.1 mL volume) that previously was equilibrated with 10 column volumes (CV) of 25 mM Na-Acetate, 12.4 mM acetic acid, pH 5.0 using a flow rate of 25 CV/h.

The antibody was collected in the flow-through fraction during the loading procedure. The antibody concentration in the collected flow through-pool was determined by absorbance (at 280 nm and extinction coefficient=1.58 (g/L)-1·cm-1). Recovery of the antibody in the flow-through fraction was above 92%. HCP (host cell protein) and HMWP (aggregates) in this fraction was reduced by a factor of 10 and 3, respectively. Leakage of protein A derivative from the capture step was likewise reduced significantly. Due to the very high load of antibody on the resin, bound monomer was displaced by HMWP and HCP (host cell protein) during load. To obtain maximum reduction of impurities, an adjustment of pH for the chromatographic process may be necessary in the range of pH 4.5-6.0. Likewise, the conductivity may be optimized in the range 0-100 mM NaCl. Levels of HCP and HMWP in sample solution and flow through fraction are shown in Table 5.

TABLE 5

| — | Volume | Conc | Yield | Monomer | HMWP | HCP | |
|---|---|---|---|---|---|---|---|
| — | [ml] | [mg/ml] | [%] | [%] | [%] | (ng/ml) | ppm |
| Sample solution | 477.8 | 5.2 | — | 98.12 | 1.88 | 3060 | 587.3 |
| Flow through fraction | 477.8 | 4.8 | 92.1 | 99.25 | 0.75 | 321 | 66.9 |

The collected CIEX flow-through pool was adjusted to pH 7.0 with 0.5 M di-sodium hydrogen phosphate. The conductivity was adjusted to 7.0 mS/cm with water. The volume of the loading solution was 825 mL, and the antibody concentration was 2.7 mg/mL. The solution was passed through a Sartobind Q-MA75 (membrane volume 2.1 mL) previously equilibrated in 35 membrane volumes (MV) of 20 mM sodium phosphate, 50 mM NaCl, pH 7.0 at a flow rate of 300 MV/h. The membrane was subsequently washed with 20 MV of 20 mM Na-phosphate, 50 mM NaCl, pH 7.0.

The antibody was collected in the flow-through pool. The antibody concentration in the pool was determined to 24.8 mg/mL. The step yield was above 99% and the reduction factor for HCPCHOP was 3. Levels of HCP and HMWP in sample solution and flow through fraction are shown in Table 6.

TABLE 6

| — | Volume | Conc | Yield | Monomer | HMWP | HCP | |
|---|---|---|---|---|---|---|---|
| — | [ml] | [mg/ml] | [%] | [%] | [%] | (ng/ml) | ppm |
| Sample solution | 825.0 | 2.7 | — | 99.25 | 0.75 | 321 | 119.3 |
| Flow through fraction | 830.3 | 2.66 | 99.5 | 99.24 | 0.76 | 99.3 | 37.3 |

### Example 18

### Purification of Anti-NKG2D Using a 2-Step Approach

The cell culture supernatant from a CHO cell culture (3.5 g/l) was filtrated and loaded on a 106 ml Protein A derivate (MabSelect SuRe) affinity column (length 11 cm) (for solvents and conditions, see Example 1). All steps were performed at room temperature. Elution was achieved with an elution buffer of 10 mM formic acid at pH 3.5. The eluted product pool was subjected to virus inactivation by adjusting pH to pH 3.6 with 0.2 M citric acid and kept at room temperature for 1 hour. Subsequently the eluate was adjusted to pH 7.0 with 0.5 M Na₂HPO₄ filtrated to remove precipitates. The solution was adjusted to 7.0 mS/cm (water or NaCl) at room temperature before loading it on a Sartobind Q SingleSep capsule (75 cm²) anion exchange membrane. An anion exchange resin may also be used. The anion-exchange membrane step was run in flow-through mode at non-binding conditions and the filtrate was finally ultra- and diafiltrated with a 50 cm² Biomax 30k membrane on an Äkta cross-flow equipment into a 10 mM Histidine buffer pH 6.2, followed by addition of Tween 80 to 0.01%. A virus filtration could be added after the anion exchange step. The end composition of the drug substance formulation was 50 mg mAb/mL, 80 g/L sucrose, 0.03% w/w Tween 80, 10 mM Histidine, pH 6.2. Results for the purification are given in Table 7. Conductivity and pH of the Q membrane step and the loading solution passed through the membrane will have to be adjusted for each mAb to achieve maximum reduction of impurities with highest possible yield. The conductivity and pH may thus vary in the range of 2-12 mS/cm (controlled by the NaCl content) and pH 5.8-8.0, respectively.

TABLE 7
Summary of Anti-NKG2D 2-step process

| | | Conductivity | Content | Yield | HMWP | HCP |
|---|---|---|---|---|---|---|
| Process step | pH | (ms/cm) | mg/ml | (%) | (%) | (ppm) |
| Cell culture | 7.0 | — | 3.5 | — | — | — |
| Affinity pool | — | — | 10.6 | 96 | 1.6 | 592 |
| Q membrane | 7.0 | 7.1 | 9.0 | 86 | 1.5 | 120 |
| pool | | | | | | |
| UF/DF filtrate | 6.2 | — | 55.7 | 95 | 1.5 | 105 |

Description of analytical methods are given in Example 9.

1. A method for purifying an antibody compound from a suspension comprising said antibody compound, the method comprising
i) bringing said suspension into contact with a Protein A derivative/analogue under conditions, wherein the Protein A derivative/analogue binds the antibody compound,
ii) washing said Protein A derivative/analogue bound antibody with a suitable buffer, and
iii) eluting said antibody compound from the Protein A derivative/analogue with a suitable buffer and collecting said antibody compound in a resulting eluate.

2. A method according to claim 1, wherein said Protein A derivative/analogue resin is MabSelect SuRe™.

3. A method according to claim 1, wherein one or more of steps i) to iii) is performed at a temperature below room temperature.

4. A method for purifying an antibody compound from a suspension comprising said antibody compound, the method comprising
i) bringing said suspension into contact with ligand having affinity for such antibody under conditions, wherein said ligand binds the antibody compound,
ii) washing said ligand bound antibody with a suitable buffer,
iii) eluting said antibody compound from the ligand with a suitable buffer and collecting said antibody compound in an eluate, and
subjecting the eluate from step iii) to cation chromatography.

5. A method according to claim 4, wherein said ligand is Protein A.

6. A method according to claim 1, wherein the eluate from step iii) is subjected to cation chromatography.

7. A method according to claim 4, wherein the eluate from step iii) is subjected to virus inactivation prior to being subjected to the cation chromatography

8. A method according to claim 4, wherein the cation chromatography is performed at a temperature below room temperature.

9. A method according to claim 1, wherein the eluate from step iii) is subjected to anion chromatography.

10. A method according to claim 9, wherein the eluate from step iii) is subjected to virus inactivation prior to being subjected to the anion chromatography.

11. A method according to claim 4, wherein the eluate from the cation chromatography is subjected to anion chromatography, wherein the eluate from the cation chromatography is optionally subjected to a virus filtration prior to being subjected to anion chromatography.

12. A method according to claim 11, wherein the eluate from the cation chromatography is subjected to a virus filtration prior to being subjected to anion chromatography.

13. A method according to claim 9, wherein the anion chromatography is performed at a temperature below room temperature.

14. A method according to claim 9, wherein said anion chromatography is performed as flow-through using a membrane or a solid resin.

15. A method according to claim 9, wherein the eluate from the anion chromatography is subjected to a virus filtration.

16. A method according to claim 1, wherein the final eluate is subjected to diafiltration and/or ultrafiltration.

17. A method according to claim 1, wherein the final eluate is formulated into a pharmaceutical preparation.

18. An antibody purifying platform, which platform comprises a method according to claim 1.

19. An antibody purifying platform suitable for purifying IgG antibodies, which platform comprises a method according to claim 1.

20. An antibody purifying platform according to claim 18, wherein said platform is used for production of therapeutic IgG antibodies.

21. A process for the industrial production of antibodies, wherein said process comprises a method according to claim 1.

22. A process according to claim 21, wherein the steps following the eluation from the Protein A derivative/analogue chromotography are performed in a continous process mode without holding tanks.
